(19) United States
(12) Patent Application Publication
(10) Pub. No.: US 2002/0177841 A1
(43) Pub. Date: Nov. 28, 2002

(54) Dilatation catheter with stiffening wire

(76) Inventors: Noreen Moloney, Galway (IE); Patrick J. Duane, Galway (IE); Thomas K. Fitzmaurice, Santa Rosa, CA (US)

Correspondence Address: MEDTRONIC AVE, INC., 3576 UNOCAL PLACE, SANTA ROSA, CA, 95403, US

(21) Appl. No.: 10/118,529

(22) Filed: Apr. 8, 2002

Related U.S. Application Data
(63) Continuation-in-part of application No. 09/136,330, filed on Aug. 19, 1998, now Pat. No. 6,368,302.
Application No. 09/136,330 is a continuation of application No. 08/387,906, filed on Jun. 29, 1995, now Pat. No. 5,823,995.
Application No. 08/387,906 is a continuation-in-part of application No. 07/934,948, filed on Aug. 25, 1992, now abandoned.
Application No. 07/934,948 is a 371 of international application No. PCT/US93/07943, filed on Aug. 24, 1993.

(51) Int. Cl. A61M 25/01

(52) U.S. Cl. 604/528; 606/194

(57) ABSTRACT

A rapid exchange dilation catheter having an elongated tubular shaft section with a guidewire port for entry of a guidewire into said distal section at an exchange joint. A stiffening wire is positioned within the elongated tubular shaft with its distal end anchored approximate the exchange joint and its proximal end freely floating within the proximal section of the catheter.

This application is a continuation in part of Ser. No. 09/136,330, filed Aug. 19, 1998, which is a continuation of Ser. No. 08/387,906, filed Jun. 28,1995 which is now U.S. Pat. No. 5,823,995, which is a continuation in part of Ser. No. 07/934,948, filed Aug. 25, 1992 and now abandoned, which is a Section 371 of PCT/US93/07943, filed Aug. 24, 1993.

### FIELD OF THE INVENTION

This invention relates to a dilatation catheter and more particularly to a monorail or rapid exchange type dilation catheter having a stiffening wire.

### BACKGROUND OF THE INVENTION

Rapid exchange dilatation catheters are commonly used in angioplasty procedures because the unique construction of such catheters enables the rapid exchange of the catheter once it is inserted into the patient. For example, U.S. Pat. No. 4,762,129 to Bonzel discloses a rapid exchange catheter having a short tube defining a guidewire lumen at the distal end of the catheter. The tube extends through the balloon from the distal end to a point proximal of the proximal end of the balloon. This tube terminates at an aperture opening to the exterior of the catheter such that most of the length of the guidewire from the balloon to the proximal end of the catheter extends exteriorly of the catheter. Rapid exchange and manipulation of the dilatation catheter is facilitated because the catheter segment contacting the surface of the guide wire is only as long as the balloon.

Although the rapid exchange catheter is designed for rapid catheter exchange, the rapid exchange catheter is known for its lack of stiffness and pushability. In the Bonzel construction, a stiffening wire extends through the catheter imparting stiffness to the catheter. However, the stiffening wire may impart stiffness in areas of the catheter where flexibility is desired, for example in the balloon. Additionally, it maybe desirable to strengthen only the proximal portion of the catheter. The Bonzel stiffening wire extending fully through the balloon does not selectively maximize stiffness of the proximal portion of the catheter while maintaining flexibility of the distal portion.

One commercial dilatation balloon catheter manufactured by Schneider (Europe) AG, uses a triple tapered stiffening wire extending through the catheter in an attempt to provide for selective, maximized stiffness of the proximal portion of the catheter while maintaining some flexibility at the distal portion of the catheter. It is believed that the distally directed tapers of this stiffening wire are selected so as to enhance flexibility from the proximal to the distal portions of the catheter. It can be desirable, however, to secure distal and proximal ends of a stiffening wire within the catheter in such positions to ensure stiffness of the proximal portion of the shaft while maintaining flexibility of the distal end and balloon.

Thus, another approach is to provide a dilatation catheter in which a stiffening wire is anchored in place at both its distal and proximal ends. The distal end terminates at and is secured in place within the catheter in the region of the entrance port for the guidewire. The proximal end of the stiffening wire is anchored proximal of the guidewire port such as at the luer fitting on the proximal end of the catheter. Alternatively, a shorter stiffening wire may be used with its proximal end secured to the interior wall of the catheter. However, in this arrangement, the stiffening wire does not maintain a coaxial orientation and may tend to orient the catheter with respect to the location of the anchor. Advancement of the catheter along the tortuous vascular anatomy may be inhibited because of the catheter's tendency to orient with respect to the catheter wall location at which the stiffening wire is anchored. Furthermore, the location at which the guidewire is secured becomes a kink point also affecting advancement of the catheter.

Thus, what is needed is a stiffening wire that provides the requisite support and can readily navigate the vascular anatomy.

### OBJECTS OF THE INVENTION

It is therefore an object of the present invention to provide a dilatation catheter of the rapid exchange type in which a stiffening wire strengthens the proximal portion of the shaft without reducing flexibility at the distal portion of the catheter and balloon.

A further object of the present invention is to provide means for securely anchoring the distal end of a stiffening wire within a rapid exchange type of the catheter at a location adjacent to the guidewire entrance port so as to impart stiffness to the proximal portion of the catheter while maintaining flexibility at the distal end.

A further object of the present invention is to provide a rapid exchange type catheter having a stiffening wire imparting rigidity to the proximal portion of the catheter and having means aiding in imparting flexibility to the distal end of the catheter and the balloon.

Yet another object of the present invention is to provide a stiffening wire secured approximate the guidewire port without compromising the flexibility of the proximal portion of the catheter.

Additional objects and advantages of the invention will be set forth in the description which follows and, in part, will be obvious from the description and advantages being realized and attained by means of the instrumentation, facts, apparatus, systems, steps and procedures particularly pointed out in the specification.

### BRIEF DESCRIPTION OF THE INVENTION

In accordance with the present invention, the balloon dilation catheter of the present invention solves the above problems by providing a rapid exchange catheter construction in which a stiffening wire is secured at both its ends in selected positions imparting maximum strength in the proximal portion of the catheter while maintaining flexibility in the distal portion of the catheter and the balloon. In accordance with the present invention, the proximal end of the stiffening wire is secured at the luer fitting while the distal end of the stiffening wire is secured at an area adjacent to the guidewire entrance port.

In one preferred embodiment, the shaft includes inner and outer tubes that define an inflation lumen. A dilatation balloon is mounted on the distal end of the outer tube and is in communication with the inflation lumen. The inner tube extends axially through the balloon. A guidewire entrance port is formed within the outer tube at a location proximal to the balloon and in communication with the portion of the inner tube extending into the balloon. A stiffening wire is positioned within the inner tube and has a proximal end secured in the proximal portion of the catheter shaft. The distal end of the stiffening wire is secured in an area adjacent the guidewire entrance port. The secured stiffening wire imparts strength to the proximal portion of the catheter shaft so as to resist buckling while maintaining flexibility at the distal portion of the catheter and balloon.

In one aspect of the invention the stiffening wire is secured at a position proximal to the guidewire entrance port. In another aspect of the present invention, the stiffening wire is secured at a position distal to the guide wire entrance port. The distal end of the stiffening wire is secured by the inner tube which is heat shrunk around the distal end of the stiffening wire. In another embodiment, the distal end of the stiffening wire is secured between the inner and outer tube by heat bonding, adhesive or other means. In still other embodiments, the distal end of the stiffening wire is secured within the inner tube by an adhesive.

In one illustrated embodiment, the outer tube is a single tube. In another embodiment, the outer tube comprises a proximal tube segment having a distal end, and a distal tube segment inserted over the distal end of the proximal tube segment.

In one embodiment, the proximal end of the catheter is formed only from the outer tube. The inner tube is secured to the outer at a location distal to the proximal end.

In still another embodiment, the proximal end of the catheter is formed from a hypotube having a distal end. The inner and outer tubes are connected to the distal end of the hypotube.

In one preferred embodiment, a substantially radiopaque coil is mounted over the inner tube within the balloon. The coil aids in imparting flexibility to the distal end of the catheter containing the balloon.

The guidewire entrance port may be formed by a transverse slit extending through the distal end of the proximal tube segment. The tube portion proximal to the transverse slit is biased inward to form the guidewire entrance port and to form a downwardly extending guidewire entrance port wall segment.

In accordance with a second preferred embodiment of the present invention, a rapid exchange balloon dilatation catheter includes a stiffening wire secured approximate the guidewire rapid exchange joint such that maximum strength is imparted in the desired area of proximal portion of the catheter while flexibility is maintained in the distal portion of the catheter and the balloon. The catheter comprises an elongated shaft with a distal section, an intermediate section, and a proximal section. A guidewire port is positioned between the intermediate section and distal section of the shaft such that the guidewire lumen extends from the catheter distal tip to a location just proximal of the balloon.

The stiffening wire may be tapered and it is secured to the distal shaft section adjacent the guidewire exchange joint. The proximal end of the stiffening wire extends through the intermediate section and into the proximal section of the catheter shaft. The proximal section of the stiffening wire is not anchored but allowed to freely float within the proximal section of the shaft. Thus, the stiffening wire supports the polymeric tubing, the exchange joint and the proximal section of the distal shaft.

### DESCRIPTION OF THE FIGURES

The foregoing and other objects and advantages of the present invention will be appreciated more fully from the following description, with references to the accompanying drawings in which:

FIG. 1 is a highly schematic plan view of the catheter of the present invention in which the outer tube is formed from a single tube;

FIG. 2 is an enlarged view of the tapered inflation luer adapted for connection to an inflation device;

FIG. 3 is another embodiment of the catheter using a hypotube at the proximal end of catheter;

FIG. 4 is an enlarged view of the embodiment of FIG. 3;

FIG. 5 is a detailed sectional view of another embodiment of the catheter having proximal and distal outer tube segments and showing the stiffening wire secured adjacent the guidewire entrance port;

FIG. 6 is a sectional view taken along line 6-6 of FIG. 5;

FIG. 7 is a sectional view taken along line 7-7 of FIG. 5;

FIG. 8 is a sectional view taken along line 8-8 of FIG. 5;

FIG. 9 is an enlarged plan view of the distal end of the stiffening wire;

FIG. 10 is a sectional view of the balloon of one embodiment of the invention showing a coil positioned within the balloon around the inner tube;

FIG. 11 is an enlarged sectional view of the proximal end of the balloon shown in FIG. 10;

FIG. 12 is an enlarged view of the catheter tip of FIG. 10;

FIG. 13 is a sectional view of another embodiment of the present invention where a guidewire lumen is parallel to and shorter than an inflation lumen;

FIG. 14 is an enlarged partial sectional view showing structure of the embodiment where the inflation and guidewire lumens are parallel to each other;

FIG. 15 is a sectional view taken along line 15-15 of FIG. 14;

FIG. 16 is a sectional view of the catheter adjacent the guidewire entrance port and showing the outer tube formed from a single tube; and

FIG. 17 is a side view of a rapid exchange catheter of the present invention;

FIG. 18 is a sectioned side view of the distal portion catheter of FIG. 17 shown prior to heat lamination;

FIG. 19 is a sectioned side view of the distal portion of the catheter of FIG. 17 shown post heat lamination;

FIG. 20 is a cross-sectional view along line 20-20 of FIG. 19;

FIG. 21 is a cross-sectional view along line 21-21 of FIG. 19;

FIG. 22 is a cross-sectional view along line 22-22 of FIG. 19; and

FIG. 23 is a cross-sectional view along line 23-23 of FIG. 19.

### DETAILED DESCRIPTION OF THE PREFERRED EMBODIMENT

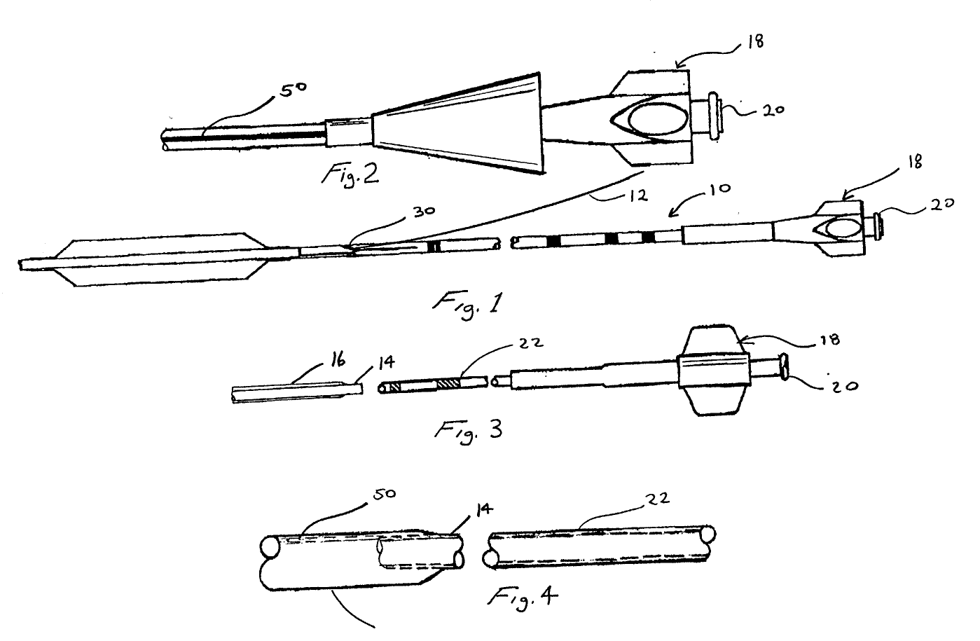

Referring now to the drawings, and more particularly FIG. 1, there is shown a preferred embodiment of the present invention. As shown in detail, the catheter, indicated generally at 10, is formed as a rapid exchange design in which the length of guidewire 12 exposed to contact with a guidewire lumen is minimized. The catheter of this embodiment includes inner and outer coaxial, polymer tubes 14, 16 (FIG. 10) forming a main catheter body shaft approximately 1.5 meters long. The proximal portion of the shaft preferably is a one tube construction, forming the inflation lumen of the catheter. The inner tube then is secured within the outer tube by conventional means, forming the two tube construction. A conventional luer fitting 18 is secured to the proximal end of the catheter shaft and includes a proximal connector 20 adapted for connection to an inflation-deflation device. In the preferred embodiment, the proximal portion of the shaft is formed from a cylindrical, polymer extrusion. In another embodiment, the proximal portion of the shaft is formed from a hypotube 22 (FIGS. 3 and 4). The more distal end of the hypotube 22 includes the polymer tube sections 14, 16 connected thereto. The inner and outer tubes 14, 16 are connected onto the distal end of the hypotube 22 (FIG. 4) in sealed relationship thereto. The hypotube 22 offers the advantage of imparting rigidity to the very proximal portion of the catheter while maintaining flexibility in the areas distal of the hypotube.

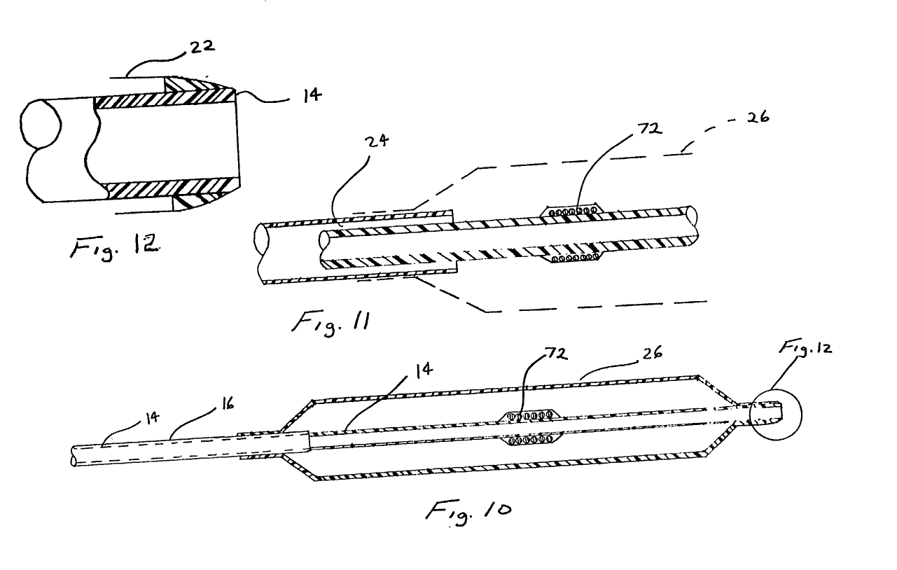

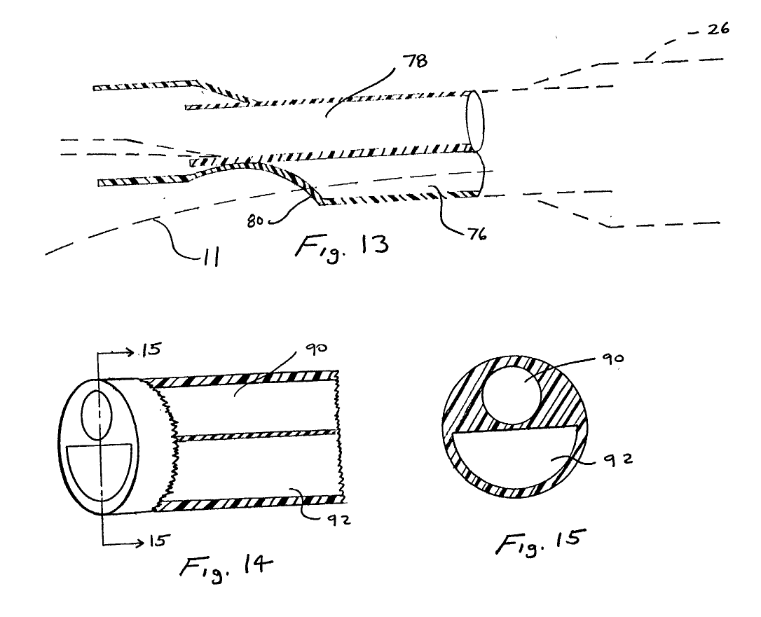

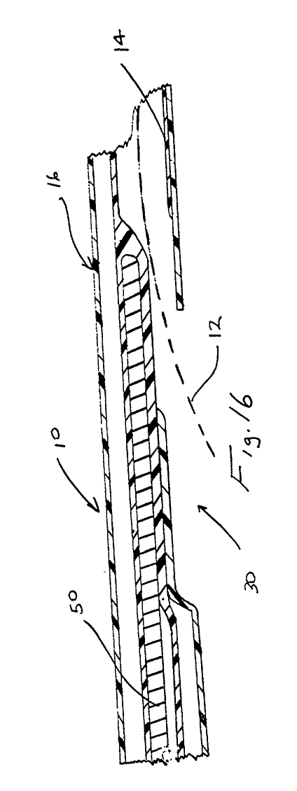

In the first embodiment, the annular space 24 between first and second tubes forms an inflation lumen. The outer tube terminates proximal to the distal end of the inner tube (FIG. 11). As shown in FIGS. 11 and 12, the proximal end of a dilatation balloon 26 is connected and sealed to the distal end of the outer tube 16. The inner tube 14 extends through the balloon 26 (FIG. 10) and is sealed at its distal end to the distal end of the balloon (FIG. 12).

The balloon 26 is formed from a variety of known compliant, semi-compliant and noncompliant balloon materials, such as polyethylene terephthalate (PET), urethane or Pebax. It is preferred that the balloon is coated with a highly lubricous, abrasion resistant coating. An example of a preferred coating is that disclosed in U.S. Pat. No. 5,077,352 to Elton, and assigned to the assignee of the present invention, C. R. Bard of Murray Hill, N.J., the disclosure of that patent which is incorporated herein by reference. As disclosed in that patent, a flexible, lubricous organic polymeric coating is formed by applying a mixture of an isocyanate, a polyol, poly (ethylene oxide), and a carrier liquid to the surface to be coated. The carrier liquid is removed and the mixture reacted to form a polyurethane coating with associated poly (ethylene oxide) giving a highly lubricous, abrasion resistant, flexible coating. However, any suitable coating may be used.

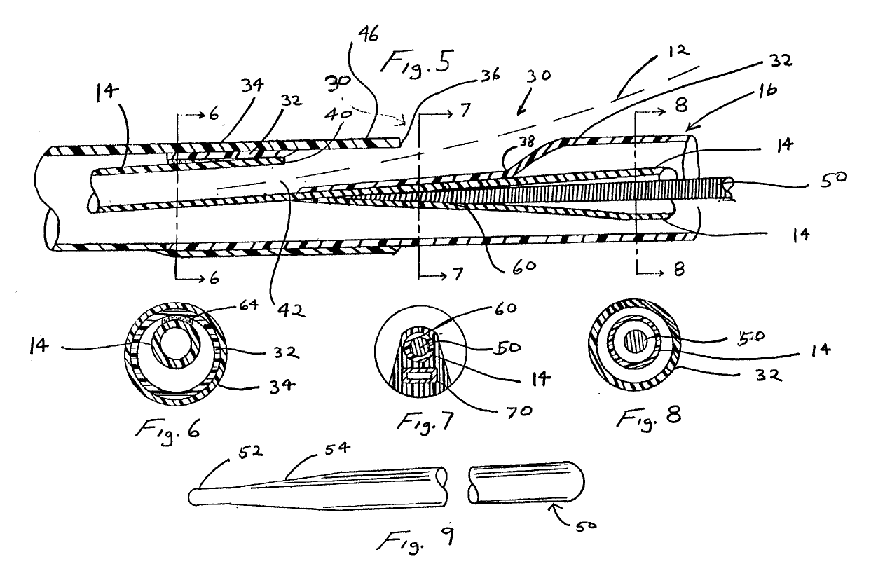

A guidewire entrance port 30 is formed proximal to the balloon (FIGS. 1 and 5). In the preferred embodiment having coaxial tubes, the guidewire entrance port 30 is formed as shown in detail in FIG. 5. In the embodiment of FIG. 5, the outer tube 16 includes a proximal tube segment 32 and a distal tube segment 34 received over the distal end of the proximal tube segment 32. The two outer tube segments 32, 34 could be adhesively bonded or heat bonded to each other. The inner tube 14 extends through both proximal and distal outer tube segments. The balloon is secured to the distal outer tube segment (not shown). In the embodiment of FIG. 1 and FIG. 16, the outer tube is a single tube and does not include proximal and distal outer tube segments as shown in FIG. 1.

The proximal tube segment 32 includes a transverse slit 36 extending through the tube 32 at a position proximal to the balloon to form the guidewire entrance port 30. In the preferred embodiment, the guidewire entrance port is positioned about twenty centimeters of the proximal end of the balloon so that the amount of guidewire contained within the proximal end of the catheter is minimal, leaving the proximal portion of the guidewire exposed and trailing external to the catheter (FIG. 1). This type of construction forms what is commonly referred to as a “rapid exchange”. The rapid exchange construction facilitates catheter exchange because the catheter tube no longer contains a substantial length of the guidewire as in conventional over-the-wire balloon dilatation catheters.

The outer tube wall segment 32 proximal to the slit 36 is folded downward (FIG. 5) and overlaps the outer surface of the inner tube 14 so as to define a smooth, downwardly extending wall surface 38 for guiding the guidewire 12 into the inner tube 14 via a slit 40 in the inner tube. The slit 40 forms a guidewire entrance opening 42 in the inner tube in which the guidewire is received. The distal outer tube segment 34 has a proximal portion 46 that partially overlaps the folded segment 38 to form a guidewire channel between the inside surface of the distal outer tube segment and the folded down portion of the proximal outer tube segment.

In accordance with the present invention, an aspect of the present invention is the use of a stiffening wire indicated generally at 50, (FIG. 9) that is secured at its proximal end to the luer fitting 18 and at its distal end to the area adjacent the guidewire entrance port 30. The stiffening wire 50 of the present invention is preferably formed from a material exhibiting the desired characteristics that maximizes the stiffness of the proximal shaft portion of the catheter. A 302 or 304 stainless steel has been found satisfactory. However, the choice of material for the stiffening wire is not limited to a stainless steel. Plastics, composite metals, and other materials also can be used as long as the selected material exhibits sufficient stiffening characteristics imparting the desired stiffness to the proximal portion of the catheter.

With a catheter that is about 150 cm long, and with a conventional length dilatation balloon, the stiffening wire is about 121 cm long. In one embodiment, the wire is about 0.016+/−0.0003 inches diameter and tapers downward to about a 0.003+/−0.0003 inch diameter straight portion 52 (FIG. 9). The tapered portion 54 is approximately 10+/−0.5 cm long, followed by the straight portion that is about 10 mm+/−0.5 cm long, followed by the straight portion that is about 10 mm+/−2 mm. When the guidewire 12 is formed of a metallic material such as the described stainless steels, the distal 9 cm of the guidewire 12 is stress relieved.

The proximal portion of the stiffening wire 50 is connected to the luer fitting 18 by conventional wire securing means used in balloon catheter technology such as an adhesive, heat bond or other conventional means. When a hypotube 22 is used, the proximal end of a metallic stiffening wire 50 is secured by brazing or other means (FIG. 4), or if stiffening wire is formed from plastic, by adhesive or other means.

In the preferred embodiment, the distal, tapered end of the stiffening wire 50 is secured to the area adjacent the guidewire entrance port 30 by heat shrinking the inner tube 14 proximal to the slit around the tapered end 54 of the stiffening wire (FIG. 5). The heat shrunk inner tube 14 provides a secure bond 60 to the stiffening wire, holding the stiffening wire in place relative to the guidewire entrance port. In the preferred embodiment, the folded-down portion 38 of the proximal outer tube segment is secured to the heat shrunk portion of the inner tube 14. The portion of the inner tube 14 distal to the slit forms a guidewire entrance opening 42. The inner tube 14 may be secured by adhesive or heat bonding to the inner wall surface of the distal outer tube segment so as to position the formed guidewire entrance opening 42 adjacent to and in close alignment with the guidewire entrance port 30. The close positional relation among the guidewire opening 42 of the inner tube, the guidewire entrance port 30, and the folded down portion of the proximal outer tube segment forms a smooth transition and passageway for the guidewire into the catheter. The smooth transition and passageway not only aids in initial guidewire placement into the catheter, but also facilitates catheter exchange.

In addition, other securing means may be used to secure the distal end of the stiffening wire to the catheter. An adhesive can be used to secure the distal end of the stiffening wire to the inside surface of the catheter. In another embodiment, the distal end of the stiffening wire may be terminated outside the inner tube, and the distal end of the stiffening wire secured between the inner tube and outer tube by heat bonding or an adhesive.

The relative position of the heat shrunk joint 60 and position of the distal end of the stiffening wire may vary somewhat relative to the guidewire entrance port 30. Positioning the joint 60 proximally relative to the guidewire entrance port 30 increases the amount of flexibility in the distal portion of the catheter. Positioning the joint 60 distally relative to the guidewire entrance port increases the stiffness of the catheter in the area adjacent the guidewire entrance port while also decreasing the flexibility of the distal portion of the catheter and balloon. A cardiologist will choose a particular construction and joint location depending on the desired flexibility and operating characteristics.

FIGS. 6, 7, and 8 are sectional views of the catheter taken along lines 6-6, 7-7, and 8-8 of FIG. 5. In the construction of FIG. 5, the outer tube is formed from proximal and distal tube segments. FIG. 6 shows the proximal outer tube segment 32 received into the distal outer tube segment 34, and an adhesive 64 securing the inner to the inner surface of the proximal tube segment. In FIG. 7, a rectangular inflation insert 70 is shown. The insert 70 maintains the continuity of the inflation lumen, which could be diminished or become closed as a result of the heat shrunk inner tube. The insert 70 is generally positioned in the area between the inner tube and the inside surface of the proximal outer tube segment. FIG. 8 shows in section the stiffening wire extending through the inner tube.

The first embodiment of the present invention also includes a coil spring 72 positioned within the balloon 26 around the inner tube 14 (FIGS. 10 and 11). The coil spring 72 ensures greater flexibility of the balloon, the distal portion of the catheter, and the tip. Additionally, the coil spring could be radiopaque to ensure that the balloon 26 can be identified under X-ray. In one embodiment, the coil is formed from 0.0025 inch spring coil material such as a gold-platinum combination. The formed coil is about 4.5 mm long. Additionally, the coil can be formed from other materials as well as other configured wire besides circular, such as rectangular. Also, the length of the coil can vary. The chosen coil parameters depend on the desired flexibility characteristics to be imparted to the distal end of the catheter.

The catheter construction of the present invention is not solely limited to the coaxial tube catheter construction described with reference to FIG. 5. As shown in FIG. 13, a two lumen or tube construction extending parallel to each other may be used. The tube or lumen 76, used for the guidewire 11 is shorter than the other inflation lumen 78. The end of the guidewire lumen 76 defines a guidewire entrance port 80. The proximal end of the stiffening wire 50 is secured to the luer fitting 18 as in the other described embodiment. The tapered distal end of the stiffening wire 50 may be secured within the other lumen and to the area adjacent the guidewire entrance port by an adhesive, heat sealing or other means.

FIGS. 14 and 15 illustrate a catheter construction where the guidewire and inflation lumens 90, 92 extend parallel to each other. Both lumens are integrally formed with each other. The distal end of the stiffening wire may be secured by the different securing means as described above.

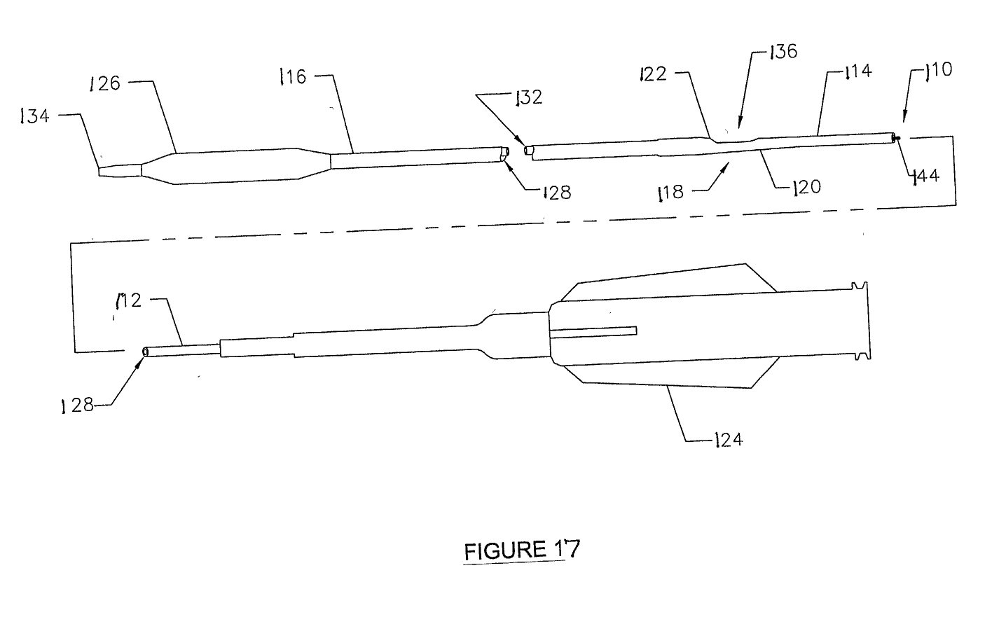

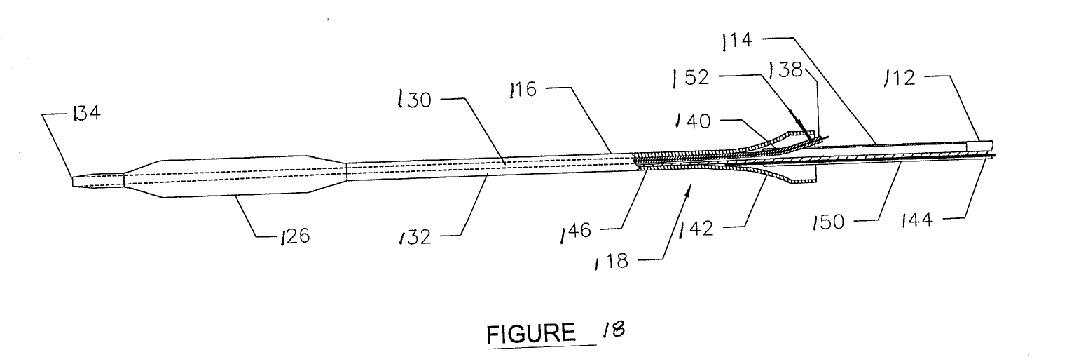

Another preferred embodiment is shown in FIGS. 17-23. A dilatation catheter, designated 110 in FIG. 17 for use in minimally invasive procedures, such as vascular procedures or other like procedures, consists of an elongated tubular shaft approximately 140 cm. long with a proximal shaft 112, a intermediate shaft 114 and a distal shaft 116. Proximal shaft 112 is preferably a PTFE coated flexitube and having a length of about 110 cm. Intermediate shaft 114 has a length of approximately 10 cm and can be any suitable material such as a polyetheramide block co-polymer. Distal shaft 116 has a length of approximately 25 centimeters and may be any suitable material. The three catheter shafts are bonded to each other through a conventional heat bond, although any other suitable bonding technique for the selected shaft materials, such as laser bonding, may be used. Exchange joint 118 is located at distal end 120 of intermediate shaft 114 where guide wire port 122 is located. A conventional luer fitting 124 is located at the proximal end of catheter 110 and balloon 126 is located at the distal end of catheter 110.

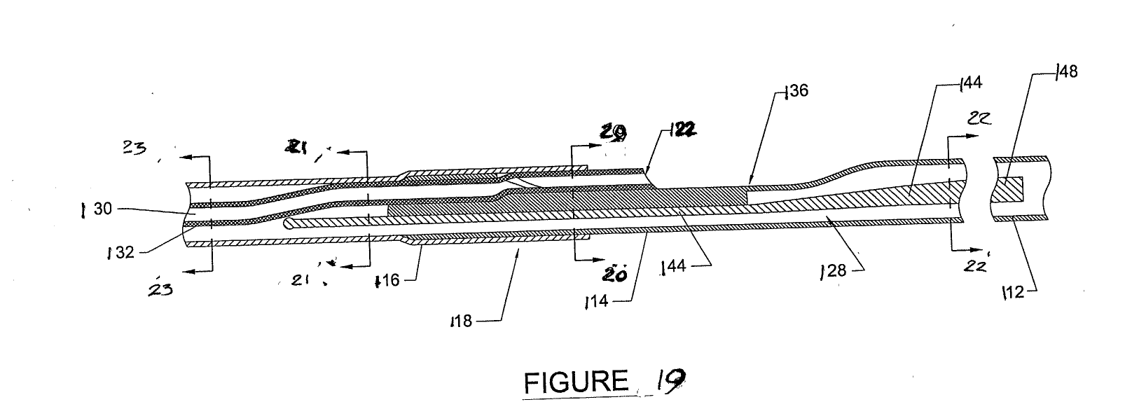

Catheter 110 includes an inflation lumen 128 that extends through catheter 110 from luer fitting 124 to balloon 126. Guide wire lumen 130 is located in a tubular member 132 extending from guide wire port 122 through distal shaft 116 and balloon 126 to the distal tip 134 of catheter 110 as seen in FIGS. 18 and 19.

Turning now to FIG. 18, catheter 110 will be described prior to the heat lamination process that bonds the components of exchange joint 118. Intermediate shaft 114 has a cut down section at its distal end to form a slot or recessed area 136. Proximal end 138 of tubular member 132 extends out of distal shaft 116, through slot 136. Distal end 140 of intermediate shaft 114 is inserted into proximal end 142 of the distal shaft 116. As seen in FIG. 18, proximal end 142 is shown flared for ease in assembling the components in preparation for the heat lamination process.

Extending through intermediate shaft 114 is stiffening wire 144. Stiffening wire 144 is preferably made of stainless steel. A tapered distal end 146 of stiffening wire 144 extends approximately 5 cm into distal shaft 116. The remaining portion extends through the length of the intermediate shaft 114 (approximately 10 cm) and into proximal shaft 112 for approximately another 10 cm. The proximal end is not anchored and freely floats within the proximal shaft. Prior to the heat lamination a flat drop mandrel 150 is placed through intermediate shaft 114 adjacent stiffening wire 144 and into distal shaft 116 as shown. This will maintain inflation lumen 138 open during the heat lamination process securing exchange joint 118. Mandrel 152 is placed into guidewire lumen 130 during the heat lamination process.

The heat lamination process is a conventional process that bonds stiffening wire 144, distal shaft 116, intermediate shaft 114 and tubular member 132 defining guidewire lumen 130, forming a laminate. The heat lamination process will cause a recessed area proximal slot 136 as seen in FIGS. 17 and 19. Tubular member 132 is preferably trimmed so that guidewire port 122 is located within the recessed area as shown in FIGS. 17 and 19.

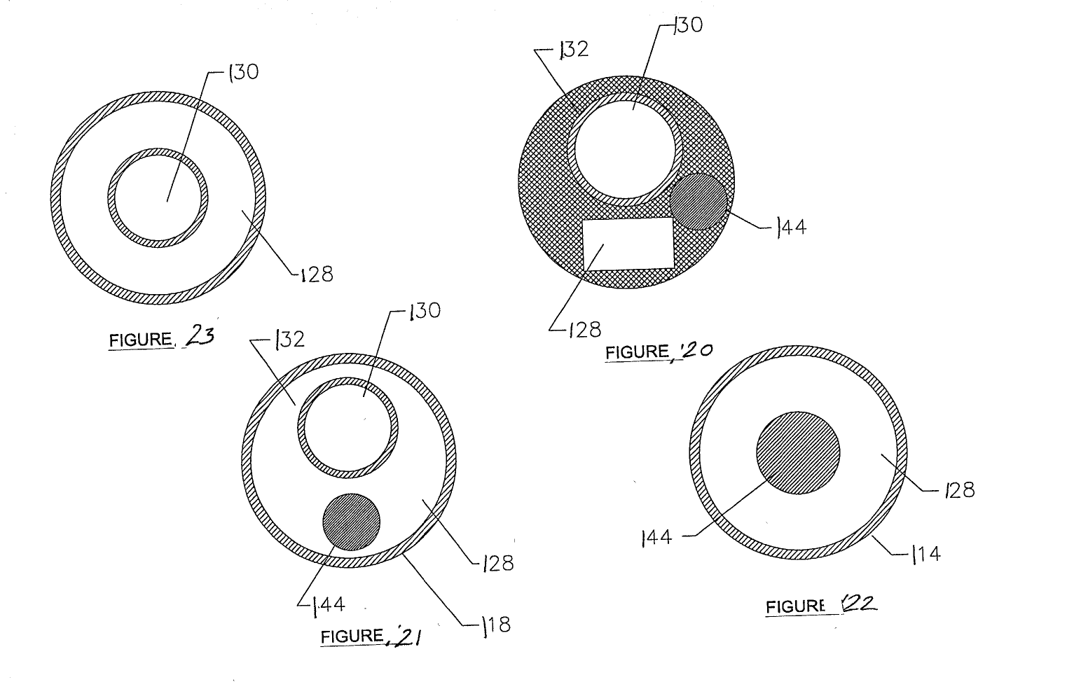

Stiffening wire 144 supports catheter 110 in the area of exchange joint 118 and provides the requisite support proximal and distal of the exchange joint. As seen in FIGS. 19 and 20, exchange joint 118 includes guidewire lumen 130, stiffening wire 144 and inflation lumen 128. The area just distal of the exchange joint, as shown in FIGS. 19 and 21, contains the guidewire lumen 130, inflation lumen 128 and stiffening wire 144. The distal portion of catheter 110 includes guidewire lumen 130 and inflation lumen 128 as shown in FIGS. 19 and 21. The proximal portion of catheter 110 consists of inflation lumen 128 along its entire length and stiffening wire 144 in approximately its distal most 10 cm as shown in FIGS. 17 and 22.

In operation, the rapid exchange catheter of the present invention provides improved performance by maximizing the stiffness of the proximal portion of the catheter, but maintaining desired flexibility of the distal portion of the catheter and balloon. Furthermore, the embodiment with the free-floating proximal end of the stiffening wire allows for unidirectional movement of the proximal shaft of the catheter enhancing the navigational characteristics of the catheter.

The foregoing embodiments and examples are illustrative and are in no way intended to limit the scope of the claims set forth herein. For example, the tubular sections of catheter 110 may be bonded with other suitable bonding techniques in addition to the heat lamination process disclosed above. Further, other suitable catheter materials may be used in addition to the coated flexitube and polyetheramide block co-polymers. The rapid exchange catheter of the present invention offers several benefits over prior art rapid exchange catheters. The use of a single lumen shaft at the proximal portion of the catheter maximizes the inflation/deflation lumen and reduces deflation times to a minimum. The coaxial distal section minimizes tip distension during balloon inflation. These and other alternatives are within the scope of the invention.

What is claimed is:

1. A catheter comprising;
an elongated tubular shaft comprising a distal section and a proximal section, said tubular shaft defining a guidewire port between said proximal section and said distal section for entry of a guidewire into said distal section; and
a stiffening wire positioned within said elongated tubular shaft and having a distal portion anchored within said tubular shaft distal section and a free proximal end extending into said tubular shaft proximal section.

2. A catheter of claim 1 wherein said stiffening wire is tapered.

3. A catheter of claim 1 wherein said stiffening wire is bonded to said tubular shaft at a location adjacent said guidewire port.

4. A catheter of claim 1 wherein said stiffening wire is thermally bonded to said tubular shaft at a location adjacent said guidewire port.

5. A catheter having a proximal end and a distal end, said catheter comprising:
an elongated tubular shaft having a guide wire port for entry of a guidewire;
a balloon attached to the catheter distal end;
a guidewire tube defining a guidewire lumen extending within said tubular shaft from said guidewire port to said distal end of said catheter,
an exchange joint located adjacent said guidewire port, said guidewire tube being bonded to said tubular shaft to retain said guidewire tube relative to said tubular shaft at said exchange joint, with a space between said guidewire tube and tubular shaft providing an inflation lumen for said balloon, and
a stiffening wire positioned within said tubular shaft, said stiffening wire secured at said exchange joint and having a free proximal end extending towards said proximal shaft end.

6. A catheter according to claim 5, wherein said stiffening wire is thermally bonded at said exchange joint.

7. A catheter according to claim 5, wherein said guidewire tube and said tubular shaft are made of thermoplastic material, and, in the region of said guidewire port, said inner tube is thermally bonded to said tubular shaft to form a fused laminate, and wherein a passageway extends through said fused laminate from the interior of said proximal end of said catheter to said inflation lumen.

8. A catheter according to claim 5 wherein said stiffening wire is tapered.
